(19) United States
(12) Patent Application Publication
(10) Pub. No.: US 2015/0342824 A1
(43) Pub. Date: Dec. 3, 2015

(54) APPARATUS FOR TREATING PROGRESSIVE MUSCLE AND BONE LOSS

(71) Applicant: Juvent, Inc., Riviera Beach, FL (US)

(72) Inventor: Peter Melott Simonson, Longboat Key, FL (US)

(73) Assignee: Juvent, Inc., Riviera Beach, FL (US)

(21) Appl. No.: 14/288,309

(22) Filed: May 27, 2014

(51) Int. Cl. A61H 23/00; A61N 5/06

(52) U.S. Cl. CPC A61H 23/00; A61N 5/0613; A61N 2005/0661; A61N 2005/064

(57) ABSTRACT

An apparatus to treat progressive bone and muscle loss by applying vibrational and radiant energy to a human body. The vibrational energy is preferably applied by a vibrational platform that transmits gentle, dynamic, repetitive vibrations to the soles of the feet. The radiant energy is preferably provided by a bed or tanning booth that emits ultraviolet radiation.

### FIELD OF INVENTION

The present invention relates to a method of treating progressive bone and muscle loss in the human body and a related apparatus. More specifically, the present invention uses both ultraviolet radiation and vibrational therapy to improve bone and muscle mass.

### BACKGROUND OF THE INVENTION

A central principle of physiology is that physiological systems strive to maintain a relatively constant internal environment or homeostasis in relation to their constantly changing external environment. Homeostatic control mechanisms are both flexible and dynamic but operate in narrow ranges required for normal function. These normal ranges, however, change over time from infancy to old age or from moment to moment, depending on the activity under way. The ability to maintain homeostasis depends on two interacting factors: (1) the status of the physiological systems involved and (2) the nature of the stress imposed. Stress is usually imposed upon the body by sudden or gradual changes in the environment. Stress may include exposure to pathogens, accidental injuries or aging. The ability to maintain such homeostatic balance varies with age, general health, and genetic makeup of the individual. If homeostatic mechanisms cannot cope with a particular external stress or internal physiological systems change, physiological values will begin to drift outside their normal range.

Disease, therefore, can be viewed as the failure to maintain those homeostatic conditions. A disease may affect a tissue, an organ or an entire system and leads to changes in the function or structure of cells throughout the body. Osteoporosis, for example, is a progressive bone disease in aging individuals characterized by a decrease in bone mass and density. It leads to the weakness of the skeleton resulting in an increased risk of fractures in the spine, wrist, hip, pelvis and upper arms. In general, osteoporosis occurs when the bone mineral density goes outside of normal range of mean peak bone mass. In addition, sacropenia is a disease that results in either reducing the number or size of muscle fibers in a body or inhibiting the ability of existing muscle fibers to contract efficiently. Sacropenia results in physical strength loss and, ultimately, to multiple fractures caused by falls.

A disease, however, can often be overcome or combated through appropriate adjustments to physiological systems. For most people, such physiological adjustments made in response to disease may lead to the reduction or, perhaps, the elimination of such diseases. Many homeostatic systems, however, become less adaptable and less efficient as people age thereby causing the significant reduction in bone mass, muscle fiber loss, lower respiratory capacity, reduced cardiac efficiency and slower kidney filtration. When individuals are exposed to such physiological stresses that their weakened or aged systems cannot tolerate, disease usually results.

Osteoporosis and sacropenia, however, can be reduced with lifestyle changes including diet, exercise and the active prevention of falls. For example, the beneficial effects of weight-bearing endurance exercise and/or exercise to strengthen muscles is scientifically well-known to improve bone and muscle strength. The musculoskeletal system relies on muscle quality and strength for support and balance. Exercise and its effect on bone and muscle maintenance is, therefore, interrelated. In young people, for example, applying or simulating high mechanical loads to bone usually promotes growth and is used to achieve a desired high peak strain of muscles. In older people, such high mechanical loads, however, can result in fracture, defeating the purpose of the exercise or treatment.

Our bodies, therefore, need micro-impacts for both our bones and muscle fibers to stay healthy. Micro-impacts are simply mechanical energy applied to the body. It is well-documented that such micro-impacts produce certain key growth and regenerative hormones in the human body. Since the human body is a dynamic biological system that responds to its environments, micro-impacts help bone and muscle tissue to stay within a certain homeostatic range. When the human body no longer receives such micro-impacts, our bones and muscles begin to weaken and soften, often resulting in osteoporosis and sacropenia. The same is true of physical inactivity which causes the physiological status of our bones and muscles to drift outside their normal range. We, therefore, need to find therapies that readjust the physiological status of our bones and muscles back into normal ranges.

It is well-known in the literature that artificial vibration applied to bone results in bone stimulation and growth. Vibration can replace the micro-impacts of mechanical energy that our bodies generally receive on a daily basis. High magnitude and high-frequency vibration is known as Whole Body Vibration (WBV). By comparison, Dynamic Motion Therapy® (DMT) is a low magnitude, low frequency and focused vibration. DMT is based on the premise that some of our muscle fibers twitch, operating at a very rapid rate to maintain our upright posture. It also assists in blood flow. These low-force muscle activities encourage bone growth and strength. When muscles fibers deteriorate with age, they may be restored with DMT. DMT can be performed, for example, by standing on a vibrating platform between 10 and 20 minutes a day. An example of such a vibrating platform is Juvent® Dynamic Motion Therapy® system. The Juvent 1000® machine is a patented biomechanical stimulation platform that vibrates bone and muscles with a low magnitude, low frequency vibration. It is a vibrating platform that a person simply stands upon.

In addition to vibrational therapy, soft bones and muscle deterioration can be treated through diet and supplements. Human bone is made of collagen and calcium phosphate, a mineral that adds strength and hardens the softer internal framework of collagen. This combination of collagen and calcium makes bones both flexible and strong. More than 99 percent of the body's calcium is contained in the bones and teeth. During one's lifetime, calcium is removed and added to the skeleton. During childhood and teenage years, new bone is added faster until a peak bone mass is reached around age 30. After that, bone reabsorption slowly begins to exceed bone formation. For women, bone loss increases significantly after menopause. When bone reabsorption occurs too quickly, osteoporosis occurs. To date, there is sufficient evidence that supplements to the diet of calcium and Vitamin D help prevent, maintain and, perhaps, restores bone mass. Furthermore, the addition of moderate amounts of calcium and Vitamin D to the diet is generally safe.

An inadequate supply of calcium over a lifetime contributes to the development of osteoporosis. Many published studies show that low calcium intake is associated with low bone mass, rapid bone loss and high fracture rates. Again, the homeostatic dietary levels of calcium get out of its normal range. It is, therefore, recommended that supplemental intake of calcium may prevent, maintain and, perhaps, restores such bone loss.

The same is true of Vitamin D. Vitamin D also plays an important role in protecting bones because it is an essential vitamin that helps the body absorbs calcium. More specifically, Vitamin D enhances the intestinal absorption of calcium which is usually ingested from the diet. Interestingly, the body can also synthesize Vitamin D within the skin. It is produced from endogenous cholesterol and is chemically transformed in the skin to Vitamin D by adequate sun exposure.

A chemical transformation occurs in human skin from exposure to ultra-violet light contained within sunlight or radiant energy. The amount of Vitamin D produced by exposure of skin to radiant energy depends, however, on the time of day, season, latitude, skin pigmentation and the amount of radiant energy. Depending on where one lives, radiant energy and, thus Vitamin D production, may decease or be completely absent during the winter. Moreover, with concerns about skin cancer, many people stay out of the sun during the summer months by covering up with clothing or using sunscreen or sun block to protect their skin. The use of sunscreen or sun block, for example, severely limits the ability of the skin to make vitamin D. For example, a SPF (sun protection factor) of 8 reduces the production of vitamin D in the skin by 95 percent. If people wish to reduce cancer risk from the sun, they will need to get vitamin D from dietary sources or supplements. Again, endogenous Vitamin D can get outside of it homeostatic normal range if skin does not receive sufficient radiant energy.

When a diet is deficient in Vitamin D or a person has inadequate sun exposure, osteomalacia or softening of the bones may occur. Such a vitamin D deficiency results in impaired bone mineralization and bone damage. As noted, a most important role of vitamin D is to maintain skeletal calcium balance by promoting calcium absorption in the intestine. It also increases the osteoclast cells which are a bone producing cells. The osteoclast is in contrast to an osteoblast which is a bone reabsorbing cell. It is again a homeostatic relationship that needs to be in a normal range between laying down new bone and taking away calcium. It is the osteoclast that maintains calcium and phosphate level for bone formation and thereby allows proper functioning of the parathyroid hormone to main serum calcium levels. When there is a vitamin D deficiency, it alters the calcium metabolism causing lower bone mineral density. The risk of reduced bone density is, of course, bone fractures. The recommended dosage and range of Vitamin D is 4,000 upper intake level (UL) per day.

To keep bones and muscles in a normal homeostatic range, the human body therefore needs adequate amounts of calcium, Vitamin D and regular exercise. When these factors are absent, bone and muscle disease occur. There is, therefore, a need to supply such essential factors and constantly readjust these factors throughout one's lifetime, especially, as people age.

### BRIEF SUMMARY OF THE INVENTION

The present invention is a method and related apparatus that provides safe, non-invasive treatment to ameliorate a significant reduction in bone mass due to the aging process. The apparatus and method adjusts such treatment according to need to assist the human body to maintain healthy bone growth and muscle strength, thereby maintaining a constant homeostatic range.

In a preferred embodiment, the apparatus provides a safe stimulation of muscle fibers in lieu of exercise by a safe and gentle vibration platform that applies mechanical energy to a body, especially, for an elder person unable to perform strenuous exercise. Additionally, the apparatus applies a safe, controlled application of radiant energy in the form of ultraviolet radiation to ensure sufficient and adequate qualities of vitamin D to enhance the uptake of dietary calcium. In combination, the apparatus creates a safe, adjustable way to maximize bone health over time that can help stop, maintain or reverse bone mass loss.

### BRIEF DESCRIPTION OF THE DRAWINGS

FIG. 1 is a perspective view showing a patient standing on the vibration device of the present invention surrounded by the ultra-violet radiation device.

FIG. 2 shows a side view of the vibration device providing the Dynamic Motion Therapy® to the patient's lower extremities.

FIG. 3 shows a perspective view of a vibration device, such as the Juvent®1000 machine produced by the Juvent/Regenerative Technology Corporation.

### DETAILED DESCRIPTION OF THE INVENTION

In its preferred form, the apparatus of the present invention combines two devices. The first device is a machine that produces low magnitude and low frequency vibrations. This device provides mechanical energy to the bones and muscles of an upright person. It is a vibration platform that transmits gentle, dynamic, specific and repetitive vertical displacements or impacts to the soles of the feet. The second device produces radiant energy to the skin to produce endogenous Vitamin D. This device is preferably an upright tanning bed or tanning booth that emits ultraviolet radiation. Such a device provides an artificial sunlight platform that assists human skin to produce Vitamin D. In combination, both devices make an apparatus that can promote bone health over time and, perhaps, stop, maintain or reverse bone and muscle mass loss.

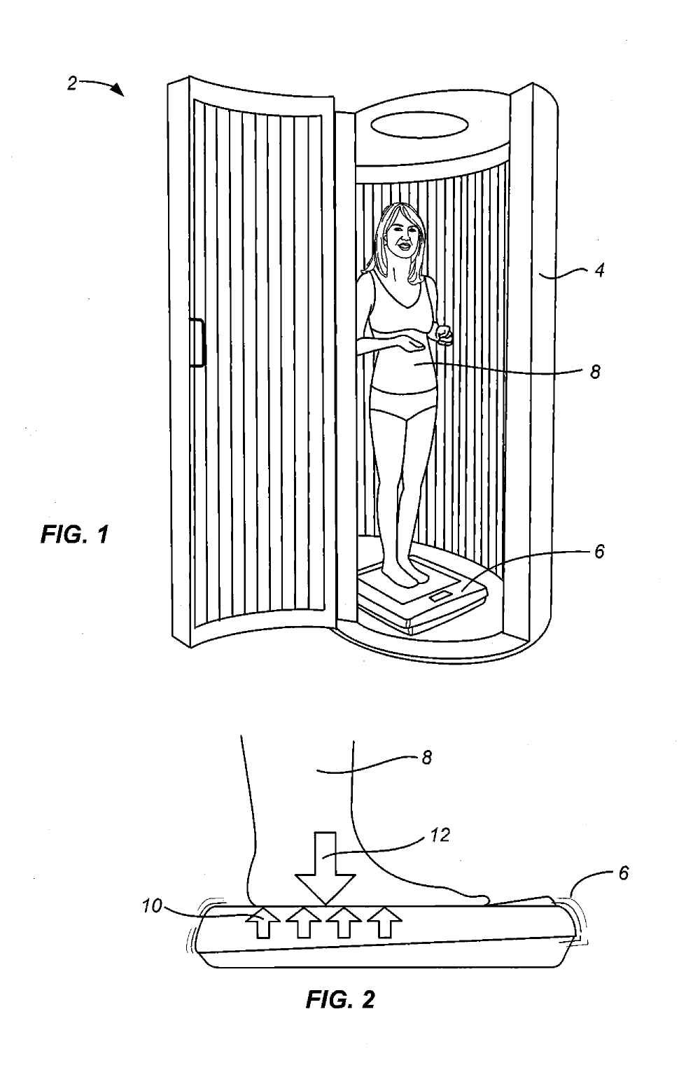

FIG. 1 shows the Apparatus 2 of the present invention as a combination of a Radiation Device 4 and a Vibration Device 6. FIG. 1 also shows a Patient 8 inside the Apparatus 2 standing upon the Vibration Device 6 and surrounded by the upright Radiation Device 4. The Radiation Device 4 can be a tanning bed, tanning booth or other source of radiant energy, electrical energy or electromagnetic frequency (EMF) energy. A tanning booth is similar to a tanning bed but the person stands while tanning, rather than lying down. While most tanning beds are horizontal, the preferred embodiment of the present invention has the Radiation Device 4 in upright position, similar to a tanning booth.

The purpose of the Radiation Device 4 is to provide the benefits of increased Vitamin D production. Commercially available tanning beds usually emit ultraviolet radiation (UVR) in two different spectrums. About 97% is ultraviolet A light (UVA) and about 3% is ultraviolet B light. UVR absorption is, however, highly variable and depends on skin types. The Fitzpatrick scale is a measure of the skin's response to UV radiation. For Fitzpatrick Type I and II (fair skin, eyes and hair) people, they can burn easily and produce maximal vitamin photosynthesis in less than 10 minutes a day. Particularly for Fitzpatrick Type I and II people, the human body can produce up to 10,000 IUs of Vitamin D with only 10 minutes of exposure to natural sunlight. Commercial tanning beds use similar UVA and UVB ratios as the sun. Such tanning bed emitters or lamps, however, vary in the intensity of UVA and UVB generation. Recently, high-intensity UVA-emitting lamps have been introduced to tan more efficiently in shorter sessions. These tanning beds have a much lower ratio of UVB to UVA and are much less effective for the purpose of Vitamin D production. The Radiation Device 4 of present invention is preferably a tanning booth that emits a higher intensity of UVB than UVA.

A study by the U.S. National Institutes of Health (Tangpricha, V., et. al.) stated that “the regular use of a tanning bed that emits Vitamin D-producing ultraviolet radiation is associated with high 25(OH)D [Vitamin D] concentrations and, thus, may have a benefit for the skeleton.” An Institute of Medicine also concluded that there is strong evidence of Vitamin D′s benefit for bone health.

The Indoor Tanning Association estimates that there are 30 million people that use some form tanning beds or booths. This is about 10 percent of the U.S. population. Among users, about 13% are teens, 20% are young adults (18-29), 13% are adults (age 30-64) and 10% are older adults (age 65+).

Promoting bone growth is important to maintain strong bones and also prevent bone fractures. Numerous different techniques, apparatuses and devices have been developed to maintain or treat bone strength. While one technique involves applying or simulating mechanical loads on bone to promote growth with the use of high frequency, high magnitude loads to the bone, this technique has been found to be unnecessary and possibility detrimental to bone maintenance.

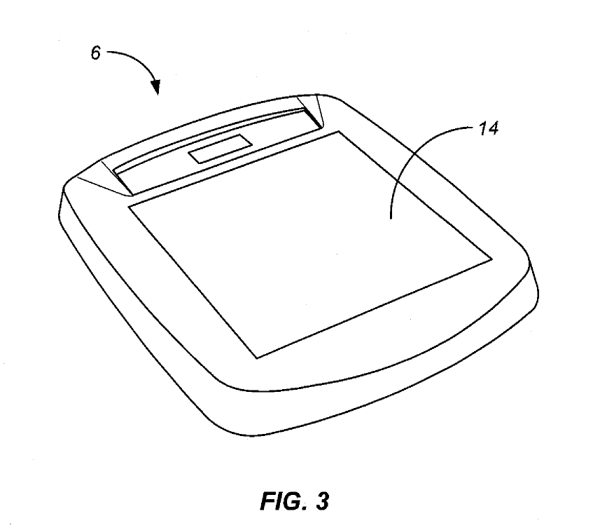

It is known in the art that low level, high frequency stress can be applied to bone, and this will result in the advantageous promotion of bone growth. FIG. 1 illustrates a Vibration Device 6 located on the floor surrounded by the Radiation Device 4 to apply such low level, high frequently vibration. As shown in FIG. 2, the purpose of the Vibration Device 6 is to support the Patient 8 on a platform that can be actuated to oscillate vertically 10. The oscillation of the platform, together with acceleration brought about by the body weight 12 of the patient, combines to stimulate bone growth. The Vibration Device 6 provides stress levels in a frequency range sufficient to prevent or reduce bone loss and enhance new bone formation.

This optimal treatment method, however, depends on using a Vibration Device 6 that measures the weight/mass of the Patient 8 and makes adjustments to the frequency of oscillation to achieve the desired therapeutic effect. One such machine for achieving this type of stress is the Vibration Device 6 disclosed in U.S. Pat. No. 7,985,191. It is known commercially as the Juvent 1000® 14 and illustrated in FIG. 3. A Vibration Device 6 such as the Juvent 1000® 14 shown in FIG. 3 preferably contains an oscillator that is positioned within the Vibration Device 6 and is configured to impart an oscillating force on the body. A capacitor assembly is preferably positioned adjacent to the oscillator for determining the mass of the Patient 8 being supported on the Vibration Device 6. Once the mass of the body is determined, the amplitude and frequency of the oscillating force is adjusted to provide a desired therapeutic treatment to the Patient 8.

In combining the Radiation Device 4 and the Vibration Device 6, the Apparatus 2 of the present invention maximizes bone health in a way that, over time, may stop, maintain or reverse bone and muscle mass loss by offering biomechanical stimulation to the bones and muscles and ultraviolet radiation to the skin to produce endogenous Vitamin D which promotes the absorption of calcium necessary for bone growth and maintenance. In an alternative embodiment, the Patient 8 stands in the Radiation Device 4 and has their bare feet grounded to the earth. While this alternative “earthing” embodiment does not provide the impact stimulation of a Vibration Device, it can allow electromagnetic energy to pass into a person's body through their feet.

The Radiation Device 4 and the Vibration Device 6 of Apparatus 2 is preferably therapeutically adjustable according to a Patient's 8 need to readjust their bone and muscle strength back into a normal range. If, for example, a Patient 8 has sufficient amounts of calcium and Vitamin D, but lacks regular exercise, the Patient 8 may stand on the Vibration Device 6 for a longer exposure to micro-impact or mechanical energy than radiant exposure from the Radiation Device 4. On the other hand, if mineral density is a greater requirement, additional exposure to radiant energy from the Radiation Device 4 may be necessary. In any event, the purpose of the present invention to reestablish bone mass and muscle strength homeostasis by imparting radiant energy and/or mechanical energy by the Apparatus 2. The Radiation Device 4 and Vibration Device 6 can be operated either continuously or intermittently.

### EXAMPLE

The invention now being generally described may be more readily understood by reference to the following example, which is included merely for purposes of illustration of certain aspects and embodiments of the present invention, and is not intended to limit the invention, as one skilled in the art would recognize from the teachings herein and the following example.

### Example 1

A Patient 8 enters a tanning salon equipped with both a Radiation Device 4 and a Vibration Device 6. Wherein the Patient 8 stands within the Radiation Device 4 and upon the Vibration Device 6, the Patient 8 and receives radiation and vibration simultaneously for an appropriate amount of time according to their therapeutic need (FIG. 1). For some patients, the radiation exposure and vibration last at least 3 minutes. In doing so, the Patient 8 acquires sufficient biomechanical stimulation, micro-impacts and mechanical energy to stimulate their bones and muscles. The present invention also delivers the required therapeutic amount of ultraviolet radiation to the skin of the Patient to produce endogenous Vitamin D.

What is claimed is:

1. An apparatus for applying radiant and vibrational energy to a human body comprising:
a radiation device; and,
a vibration device;

2. The apparatus of claim 1 wherein said radiation device emits ultraviolet radiation.

3. The apparatus of claim 1 wherein said vibration device produces low level, high frequency vibration to bone and muscle.

4. The apparatus of claim 1 wherein said radiation device and vibration device are used simultaneously.

5. The apparatus of claim 1 wherein said radiation and vibration devices operate continuously.

6. The apparatus of claim 1 wherein said radiation and vibration devices operate intermittently.

7. The apparatus of claim 1 wherein said radiation and vibration devices can be varied depending upon the physiology of the person using them.

8. The apparatus of claim 1 wherein said radiation device is a tanning booth.

9. The apparatus of claim 1 wherein said vibration device is a vibrating platform.

10. A method of improving health comprising the steps of:
having a person stand on a vibrating device in a booth providing radiant energy;
using the vibrating platform to stimulate the person's legs and using the booth to expose the person to radiant energy.

11. The method of claim 10 wherein said radiation exposure and vibration last at least 3 minutes.

12. The method of claim 10 wherein said radiation exposure and vibration are continuous.

13. The method of claim 10 wherein said radiation exposure and vibration operate intermittently.

14. The method of claim 10 wherein said radiation exposure and vibration regimen is variable.

15. The method of claim 10 wherein said radiation exposure and vibration can be varied depending upon the physiology of the person using them.

16. The apparatus of claim 10 wherein said length of radiation exposure can be reduced by using high intensity UVA-emitting lamps.
